(19) United States
(12) Patent Application Publication
(10) Pub. No.: US 2009/0068469 A1
(43) Pub. Date: Mar. 12, 2009

(54) FILM-FORMING COMPOSITION FOR HARD CAPSULES COMPRISING FISH GELATIN AND ITS PREPARATION METHOD

(76) Inventors: Hyun-Jin Park, Seoul (KR); Joon-Bum Yi, Seoul (KR); Ho-Jae Bae, Namyangju (KR); Young-Teck Kim, Incheon (KR); Dong-Su Cha, Seoul (KR)

Correspondence Address: JHK LAW, P.O. BOX 1078, LA CANADA, CA, 91012-1078, US

(21) Appl. No.: 12/162,894

(22) PCT Filed: Apr. 23, 2007
(86) PCT No.: PCT/KR07/01963

§ 371 Date: Jul. 31, 2008

(30) Foreign Application Priority Data
Apr. 24, 2006 (KR) 10-2006-0036782

(51) Int. Cl. C12N 9/10; C12N 11/02; B32B 5/16; B28B 1/14

(52) U.S. Cl. 428/402.2; 435/193; 435/177; 264/299

(57) ABSTRACT

The present invention relates to a film-forming composition for hard capsules comprising fish gelatin and its preparation method, and more particularly to a film-forming composition for hard capsules comprising fish gelatin using transglutaminase, the composition being able to be dried at 15 -25° C., and its preparation method. In the present invention, the problems caused due to the characteristics of fish gelatin having a low gelling temperature were solved by employing transglutaminase in the preparation of hard capsules using fish gelatin. Accordingly, the composition according to the present invention, its preparation method and the hard capsules and films comprising the composition may be useful to apply to a variety of industrial fields such as medicines, foods, etc.

### TECHNICAL FIELD

The present invention relates to a film-forming composition for hard capsules comprising fish gelatin and its preparation method, and more particularly to a film-forming composition for hard capsules comprising fish gelatin using transglutaminase, the composition being able to be dried at 15-25° C., and its preparation method.

### BACKGROUND ART

Gelatin is a kind of derived proteins obtained by treating collagen, which is present in skins, muscles and bones of animals, with acid or alkaline solutions and extracting the collagen with hot water. The gelatin has been used as a major material of the capsule due to its excellent film-forming ability, pliability and solubility similar to human body since it was developed for the first time as a capsule by F. A. B. Mothes in 1834. However, a large number of vegetarians and religionists have been reluctant to use capsules made of gelatin because the gelatin is one of animal components. In particular, a risk of animal protein products has been highly issued as bovine spongiform encephalopathy (BSE) broken out in the United Kingdom in 1986. Since the outbreak of the epidemics, there have been limitations in use of the gelatin. Due to the disadvantages of such an animal gelatin, a capsule made of a material that can substitute for the animal gelatin needs to be developed.

Fish gelatin is one of materials used as new raw materials in order to solve the above problems. The fish gelatin is similar to pig skin or bovine bone gelatins, which have been used as a raw material of the hard capsules, in aspect of the chemicophysical properties such as viscosity, strength, isoelectric point, etc., but it is uniquely different to the pig skin or bovine bone gelatins in aspect of a gelling temperature which is one of the important characteristics of the gelatin. A gelling temperature of mammalian gelatin is approximately 26° C., whereas a gelling temperature of fish gelatin is less than 20° C., which is lower than that of the mammalian gelatin. The reason that the fish gelatin has a lower gelling temperature than the mammalian gelatin as described above is that the fish gelatin has lower contents of proline and hydroxyproline than those of the mammalian gelatin. The proline and hydroxyproline are amino acids and included at an average content of 9% in the fish gelatin, while they are included at an average content of approximately 13-15% in the mammalian gelatin. Such a low gelling temperature serves as one of big problems in the process of manufacturing hard capsules using the fish gelatin. This is why, in the preparation of hard capsules using conventional animal gelatins, a gelatin solution made of the animal gelatin is sustained intact in the steps of immersing a capsule molding pin in a composition and drying the molding pin immersed in the composition, whereas a good sum of a gelatin solution made of the fish gelatin streams down a molding pin, resulting in poor capsules. In order to solve the above problems, Korean Patent Publication No. 10-2003-0067241 discloses a process for preparing fish gelatin wherein pectin is used at a content 0.5-1.0% of the total weight as a gelling agent and a molding pin immersed in the solution is cooled to a temperature of 20° C. or below so as to enhance viscosity of a fish gelatin solution. However, the patent has a problem that the process is complex and ineffective in aspects of the time and cost since an aqueous solution for preparing a fish gelatin capsule should be necessarily cooled in order to prepare hard capsules comprising fish gelatin.

Meanwhile, transglutaminase has characteristics to stabilize tissues and intracellular matrices by catalyzing binding of proteins and peptides in the presence of calcium to stimulate cross-linking of the proteins, and it is one of enzymes that are widely distributed over a variety of organs, tissues and body fluids of higher animals. Due to the above-mentioned characteristics, the transglutaminase has been used in protein-containing foods on a variety of applications such as manufacture of boiled fish paste and its improvement in quality, manufacture of bean curd, improvement in quality of processed meats, a gluing property of fishes and meats, etc. However, there is no example using the transglutaminase for the film-forming composition for hard capsules using fish gelatin.

Accordingly, the inventors made ardent attempts to solve the problems caused by the characteristics of the fish gelatin having a low gelling temperature in preparing hard capsules using fish gelatin, and then found a film-forming composition for hard capsules comprising fish gelatin using transglutaminase, the composition being able to be dried at 15-25° C., and its preparation method. Therefore, the present invention was completed on the basis of the facts.

### DISCLOSURE OF THE INVENTION

### Technical Problem

Accordingly, the present invention has been made to solve the above-mentioned problems in the prior art, and an object of the present invention is to provide a film-forming composition for hard capsules comprising fish gelatin.

Also, another object of the present invention is to provide a method for preparing a film-forming composition.

Still another object of the present invention is to provide a method for preparing hard capsules comprising fish gelatin using the film-forming composition.

Yet another object of the present invention is to provide a film for hard capsules and hard capsules comprising the film-forming composition.

### Technical Solution

In accordance with an aspect of the present invention, there is provided a film-forming composition for hard capsules comprising fish gelatin, the composition being prepared by mixing 100 ml of distilled water with 20-70% by weight (w/v) of fish gelatin on the basis of 100 ml of the distilled water, adding 5-25% by weight of a plasticizer on the basis of the total weight of the fish gelatin and adding 0.1-5.0 units of transglutaminase.

In accordance with another aspect of the present invention, there is provided a method for preparing a film-forming composition for hard capsules, the method including:

(a) mixing fish gelatin with a distilled water at 50-90° C. and adding a plasticizer to the resultant mixture to dissolve the fish gelatin for 8-24 hours;

(b) cooling the solution prepared in the step (a) to 45-55° C. and adjusting pH of the solution to pH 6-8;

(c) adding transglutaminase to the solution prepared in the step (b) and stirring the solution at 45-55° C. for 30-160 minutes; and

(d) heating the solution prepared in the step (c) to 90-100° C. for 5-20 minutes and cooling the solution to 30-40° C.

In accordance with still another aspect of the present invention, there is provided a method for preparing hard capsules comprising fish gelatin, the method including:

immersing a molding pin in the film-forming composition for hard capsules prepared according to the method for preparing a film-forming composition

as defined in claim 3; and

drying the molding pin at 15-25° C.

In accordance with yet another aspect of the present invention, there is provided a film for hard capsules and hard capsules comprising the film-forming composition.

Hereinafter, the present invention will be described in detail.

It is characterized in that the present invention provides a film-forming composition for hard capsules comprising fish gelatin using transglutaminase, the composition being able to be dried at 15-25° C., and its preparation method for the first time.

The present invention provides a film-forming composition for hard capsules comprising fish gelatin, the composition containing fish gelatin, a plasticizer, transglutaminase and distilled water.

The composition according to the present invention includes 20-70% by weight (w/v) of fish gelatin, based on 100 ml of the distilled water. If the content of the fish gelatin is less than 20% by weight (w/v) on the basis of 100 ml of the distilled water, a film immersed in a molding pin may be easily broken due to its very thin thickness. On the contrary, if the content of the fish gelatin exceeds 70% by weight (w/v) on the basis of 100 ml of the distilled water, the capsules are thick after their drying, resulting in difficulty in the assembly of the capsules.

Also, the film-forming composition according to the present invention includes 5-25% by weight of a plasticizer, based on the total weight of the fish gelatin. If the content of the plasticizer is less than 5% by weight on the basis of the total weight of the fish gelatin, the capsules may be all easily split or broken in the procedure of separating the dried hard capsules comprising fish gelatin from the molding pin since the prepared hard capsules have a high hardness. On the contrary, if the content of the plasticizer exceeds 25% by weight on the basis of the total weight of the fish gelatin, the hard capsules may not be sustained within their shape and may also be all crumpled in the procedure of separating the capsules from the molding pin since the prepared capsules are extremely soft. The plasticizer used in the present invention may be used without any limitation if it is generally used for preparing a capsule, and it is preferably selected from the group consisting of glycerol, sorbitol, polyethylene glycerol (PEG), mannitol and mantitol, and they may be used alone or in combinations thereof. Glycerol may be more preferably used as the plasticizer.

The film-forming composition according to the present invention includes 0.1-5.0 units of transglutaminase. A content of the transglutaminase has the highest effect on the preparation of the capsules. If the content of the transglutaminase is less than 0.1 units, the capsules are not properly formed at 15-25° C. On the contrary, if the content of the transglutaminase exceeds 5 units, it is impossible to prepare hard capsules since an aqueous fish gelatin solution is entirely gelled within 30 minutes. The transglutaminase is preferably added at a state of aqueous solution. For example, the transglutaminase may be added after the transglutaminase is dissolved at an amount of 0.1-5.0 units in 1-10 ml of water.

The composition according to the present invention is preferably prepared by mixing fish gelatin with distilled water, adding and dissolving a plasticizer in the resultant mixture and adjusting temperature and pH of the resultant mixture, followed by adding transglutaminase to the resultant mixture. If the transglutaminase is added prior to the plasticizer, the plasticizer may be separated from the mixture without its binding to the mixture by the reaction of the transglutaminase with the fish gelatin. Also, if the transglutaminase is added without adjusting pH of the fish gelatin, an enzyme reaction may not appear easily since the fish gelatin and the transglutaminase are the most active at the pH range of 4 and 6 to 8, respectively. Accordingly, it is the most preferred to prepare a film-forming composition for hard capsules comprising fish gelatin according to the method.

The present invention provides a method for preparing a film-forming composition for hard capsules comprising fish gelatin according to the present invention. The preparation method of a composition according to the present invention will be described in more detail, as follows.

First, fish gelatin is mixed with distilled water having a temperature of 50-90° C., and then a plasticizer is added to the resultant mixture and dissolved for 8-24 hours (Step (a) of the present invention).

Then, the mixed solution of the distilled water, the fish gelatin and the plasticizer is cooled to a temperature of 45-55° C., preferably 50-55° C., and then adjusted to pH 6-8 (Step (b) of the present invention). If the temperature of the cooled mixed solution is less than 45° C., the enzyme reaction may not occur easily due to the lower reaction temperature than the optimum reaction temperature of the enzymes, resulting in excessive time in forming a final solution. On the contrary, if the temperature of the cooled mixed solution exceeds 55° C., activity of the enzymes may be inhibited due to the higher reaction temperature than the optimum reaction temperature of the enzymes, resulting in hindrance of the desirable activity of the enzymes. Also, if the pH of the mixed solution after cooling is less then pH 6 or exceeds pH 8, the activity of the transglutaminase is deteriorated, and therefore the enzyme reaction may not occur as much as it is required for the gelatin solution.

The transglutaminase is added to the mixed solution prepared as described above, and then reacted at 45-55° C. while stirring (Step (c) of the present invention). Also, the stirring time may be varied according to the amount of the added fish gelatin, but it is preferably 30-160 minutes. If the stirring time after the addition of the transglutaminase is less than 30 minutes, the capsules may not be properly formed since the molding pin is not immersed in the gelatin solution to the extent that is suitable for physical properties of the gelatin solution because of the short enzyme reaction. On the contrary, if the stirring time is longer than 160 minutes, a tailing phenomenon may occur in the procedure of immersing the molding pin in the aqueous fish gelatin solution and pulling out the molding pin from the aqueous fish gelatin solution, and therefore it is difficult to produce high-quality capsules (see FIG. 8). Accordingly, the stirring time may be adjusted within the range of 30-160 minutes, depending on the amount of the added transglutaminase.

After the addition of the transglutaminase, if the stirring reaction is completed the solution is heated to a temperature of 90-100° C. for 5-20 minutes, and then cooled to 30-40° C. (Step (d) of the present invention). After the above reaction, the mixed solution is heated to suppress activity of the transglutaminase, and then cooled to a temperature of 30-40° C. which is suitable to prepare hard capsules.

The composition containing the transglutaminase may be dried at 15-25° C. without a cooling system according to the above-mentioned method, thereby to prepare hard capsules.

Accordingly, the present invention provides a method for preparing hard capsules using the film-forming composition for hard capsules comprising fish gelatin. The hard capsules may be preferably prepared by employing an immersion coating technique using a molding pin which is generally used in the field of preparing hard capsules. That is to say, the technique may include steps of immersing a molding pin in the film-forming composition for hard capsules prepared according to the preparation method of the hard capsules according to the present invention; and drying the molding pin at 15-25° C. The drying step is preferably carried out for 12-24 hours. Because the method for preparing hard capsules using the film-forming composition for hard capsules comprising fish gelatin of the present invention does not undergo the cooling process, the method according to the present invention is more effective in aspects of the time and cost than the conventional methods for preparing a capsule comprising a step of drying a composition using a cooling system (Korean Patent Publication No. 10-2003-0067241).

In addition, the present invention provides hard capsules and a film comprising the film-forming composition according to the present invention. The hard capsules and the film may be prepared according to the conventional method known in the art. The hard capsules may be prepared according to the method as described above, and the film may be prepared using a film casting machine. The preparations of the hard capsules and the film are preferably carried out by maintaining the composition of the present invention within the temperature range of 30-40° C. If a cooling temperature of the mixed solution is less than 30° C., a capsule pin is ununiformly immersed in the mixed solution, and therefore a surface of the capsule may not be uniform after drying the mixed solution. On the contrary, if the cooling temperature of the mixed solution exceeds 40° C., the capsule pin is slightly coated with the mixed solution when it is immersed in the mixed solution, and therefore the resultant film may be broken or crushed due to its thin thickness when it is separated from the pin after drying the mixed solution. The hard capsules and the film comprising the composition according to the present invention shows uniform thickness distribution over the entire area without any of splits or breaks, and have excellent physical properties such as tensile strength, elongation, oxygen permeability, moisture permeability, thermal property, colors and opacity.

### ADVANTAGEOUS EFFECTS

According to the present invention, the problems caused due to the characteristics of fish gelatin having a low gelling temperature were solved by employing transglutaminase in the preparation of hard capsules using fish gelatin. The method for preparing hard capsules using the film-forming composition for hard capsules comprising fish gelatin according to the present invention is more effective in aspects of the time and cost than the conventional methods since the film-forming composition of present invention may be dried at 15-25° C. Accordingly, the composition according to the present invention, its preparation method and the hard capsules and film comprising the composition may be useful to apply to a variety of industrial fields such as medicines, foods, etc.

### BRIEF DESCRIPTION OF THE DRAWINGS

The foregoing and other objects, features and advantages of the present invention will become more apparent from the following detailed description when taken in conjunction with the accompanying drawings.

FIG. 1 is a photographic diagram showing hard capsules which are prepared if a content of fish gelatin is less than 20% by weight (w/v) on the basis of 100 ml of distilled water.

FIG. 2 is a photographic diagram showing hard capsules which are prepared if a content of fish gelatin exceeds 70% by weight (w/v) on the basis of 100 ml of distilled water.

FIG. 3 is a photographic diagram showing hard capsules which are prepared if a content of a plasticizer is greater than 30% by weight on the basis of the total weight of the fish gelatin.

FIG. 4 is a photographic diagram showing hard capsules which are prepared if a content of a plasticizer is greater than 25% by weight on the basis of the total weight of the fish gelatin.

FIG. 5 is a graph showing viscosities of a fish gelatin solution measured according to the reaction time and concentration of transglutaminase.

♦: 0.1 units of Transglutaminase Added

▪: 1 units of Transglutaminase Added

▴: 5 units of Transglutaminase Added

: 10 units of Transglutaminase Added

*: Inactivated Transglutaminase Added

x: FG only

FIG. 6 is a graph showing cross-linking rates of a fish gelatin solution measured according to the reaction time of the transglutaminase.

▪: Viscosity

◯: Cross-linking Rate

FIG. 7 is a photographic diagram showing a lump that is not prepared in a form of hard capsules if the content of the transglutaminase exceeds 5 units.

FIG. 8 is a photographic diagram showing hard capsules which are prepared if the transglutaminase is reacted for more than 160 minutes to prepare hard capsules, wherein arrows represent tailing phenomena occurring in the prepared hard capsules.

FIG. 9 is an SEM photograph showing a film prepared in the present invention, the film being magnified up to 25,000 times.

FIG. 10 is a photographic diagram showing hard capsules prepared in the present invention.

### BEST MODE FOR CARRYING OUT THE INVENTION

Hereinafter, preferred embodiments of the present invention will be described with reference to the accompanying drawings.

However, it should be understood that the detailed description and specific examples, while indicating preferred embodiments of the invention, are given by way of illustration only, since various changes and modifications within the spirit and scope of the invention will become apparent to those skilled in the art from this detailed description. Units of separate components used in the present invention is represented by the weight of dry solids (dry basis), unless described otherwise herein.

### EXAMPLES

### Example 1

### Determination of Amount of Fish Gelatin for Hard Capsules

In order to determine an amount of fish gelatin suitable for preparation of hard capsules, hard capsules and films including various contents of fish gelatin were prepared by the inventors, as listed in the following Table 1. First, different amounts of fish gelatin (Norland Co, USA) were mixed with distilled water, and glycerol (Junsei, Japan) was added to the resultant mixtures, and the mixed solutions were put into a laboratory stirrer, and then the fish gelatin was dissolved at 60° C. for 4 hours at a rotary speed of 150 rpm to prepare aqueous fish gelatin solutions. Subsequently, the prepared aqueous fish gelatin solutions were cooled to 50° C., and then pH of the mixed solutions was adjusted to pH 7 by adding 1M NaOH to the mixed solution in drop. And, transglutaminase was added to 5 ml of distilled water and vortexed for 20 seconds, and then kept at 15-25° C. for 10 minutes. Each of the fish gelatin aqueous solutions was added to the aqueous transglutaminase solution prepared as described above, and then stirred at 50° C. for 30 minutes at a rotary speed of 150 rpm. The prepared solutions were heated for 20 minutes to 95° C. and cooled to 35° C.

TABLE 1
Determination of Amount of Fish Gelatin suitable for
Preparation of Hard Capsules

| | Contents | | | | | | | |
|---|---|---|---|---|---|---|---|---|
| Fish Gelatin | 20 | 25 | 30 | 35 | 40 | 45 | 50 | 70 |
| (wt % (w/v)) | | | | | | | | |
| Glycerol (wt %) | 10 | | | | | | | |
| Transglutaminase | 0.4 | 0.5 | 0.6 | 0.7 | 0.8 | 0.9 | 1 | 1.4 |
| (units) | | | | | | | | |

| | |
|---|---|
| Distilled Water | 100 |
| (ml) | |

Contents of the added glycerol are based on the total weight of the fish gelatin.

The hard capsules and the films were prepared using the film-forming solution prepared as described above. First, a molding pin for hard capsules was immersed in the solution while maintaining the film-forming solution to the temperature range of 35° C. Then, the molding pin was cooled for 10-20 seconds using a motor fan, and dried at 15-25° C. for 12 hours. After the drying process, the capsule was separated from the molding pin and cut into a suitable size, and then a head and a body of the capsule were assembled directly.

Also, the film was prepared using a film casting machine (PI-1210 Filmcoater, Tester Sangyo Co., Japan). The film-forming solution was cast onto a teflon-coated glass plate (25 cm×35 cm). At this time, a speed of a film applicator was 10-15 mm/sec. Then, the glass plate was dried for 12 hours in a constant temperature & humidity chamber adjusted to RH 50% and 25° C. Then the resultant film was separated from the glass plate.

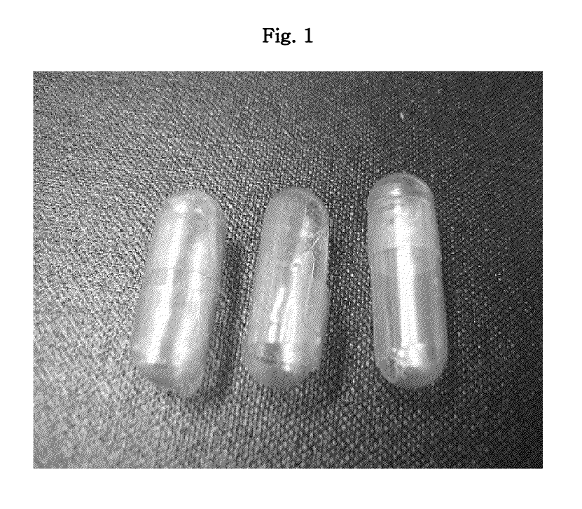

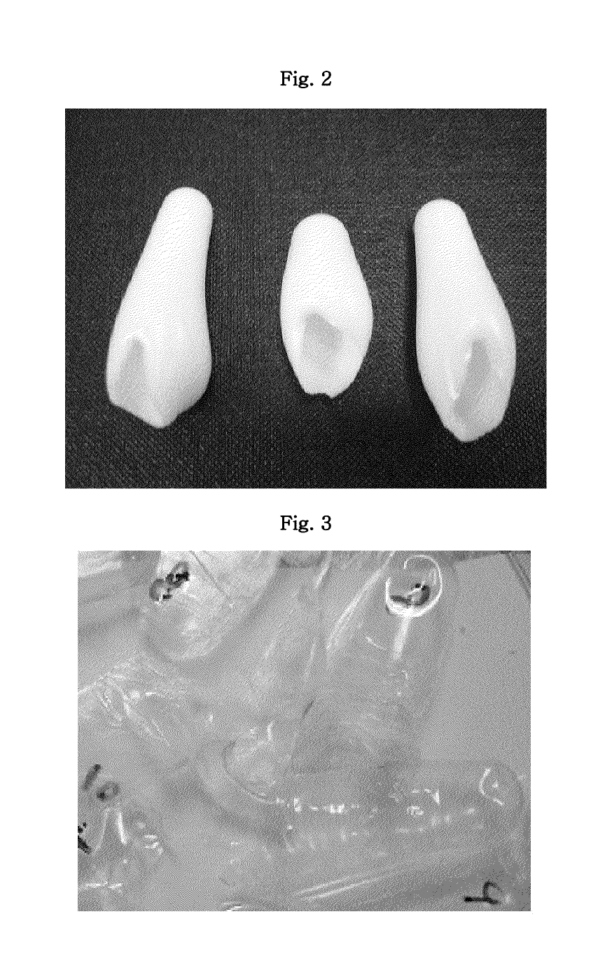

The capsule and the film prepared as described above were measured for the shape and physical properties. As a result, it was revealed that the film immersed in the molding pin is easily broken due to its very thin thickness if the content of the fish gelatin is less than 20% by weight (w/v) on the basis of 100 ml of the distilled water (see FIG. 1). On the contrary, it was revealed that the capsules are thick after their drying, resulting in difficulty in the assembly of the capsules, if the content of the fish gelatin exceeds 70% by weight (w/v) on the basis of 100 ml of the distilled water (see FIG. 2). Accordingly, it was seen that the content of the fish gelatin, which is suitable for the preparation of the hard capsules, ranges from 20 to 70% by weight (w/v), based on 100 ml of the distilled water.

### Example 2

### Determination of Amount of Plasticizer for Hard Capsules

In order to determine a content of a plasticizer suitable for preparation of hard capsules, the hard capsules including various contents of a plasticizer according to the content of the fish gelatin were prepared in the same manner as described in the Example 1 by the inventors, as listed in the following Table 2. At this time, glycerol was used as the plasticizer, and the content of the fish gelatin was 40% by weight (w/v) on the basis of 100 ml of the distilled water, which is within the optimum content range of the fish gelatin determined in the Example 1.

TABLE 2
Determination of Amount of Plasticizer suitable for
Preparation of Hard Capsules

| | Contents | | | | | | |
|---|---|---|---|---|---|---|---|
| Fish Gelatin (wt % (w/v)) | 40 | | | | | | |
| Glycerol (wt %) | 5 | 7 | 10 | 15 | 20 | 25 | 30 |

| | |
|---|---|
| Transglutaminase (units) | 0.8 |
| Distilled Water (ml) | 100 |

Contents of the added glycerol are based on the total weight of the fish gelatin.

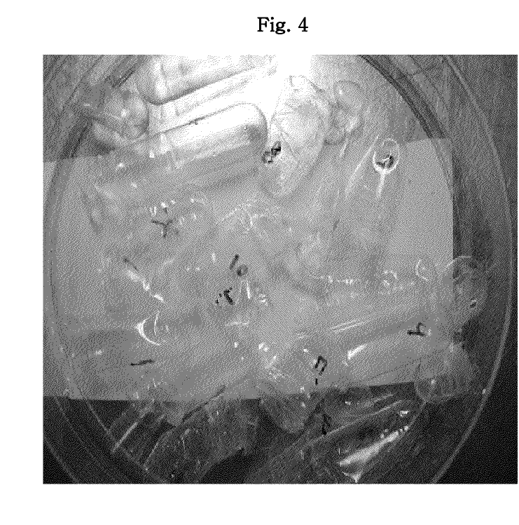

The hard capsules prepared as described above were measured for the shape and physical properties. As a result, it was revealed that, if the content of the plasticizer is less than 5% by weight on the basis of the total weight of the fish gelatin, the hard capsules may be all easily split or broken in the process of separating the dried hard capsules comprising fish gelatin from the molding pin since the prepared hard capsules have a high hardness (see FIG. 3). Meanwhile, it was revealed that, if the content of the plasticizer exceeds 25% by weight on the basis of the total weight of the fish gelatin, a capsule pin is incompletely immersed in the mixed solution since viscosity of the solution is lowered during the preparation of the capsules, resulting in difficulty in preparing capsules (see FIG. 4). Accordingly, it was confirmed that the content of the plasticizer suitable for the preparation of the hard capsules ranges from 5 to 25% by weight, based on the total weight of the fish gelatin

### Example 3

### Determination of Amount and Reaction Time of Transglutaminase for Hard Capsules

### 3-1. Effect of Transglutaminase on Viscosity and Cross-Linking Rate of Fish Gelatin Solution

In order to confirm an effect of the transglutaminase on viscosity and cross-linking rate of the film-forming composition comprising fish gelatin, experiments were carried out for physical properties, as follows.

### a) Viscosity Measurement

The viscosity of the film-forming composition comprising fish gelatin was measured according to the reaction time and contents of the transglutaminase. 40% by weight (w/v) of the fish gelatin was mixed with the 100 ml of the distilled water, and the 4 g of the plasticizer was added to the resultant mixture and stirred for 4 hours to prepare a fish gelatin solution. Then, 0.4 units of transglutaminase was added to 5 ml of distilled water and vortexed for 20 seconds, and then kept at 15-25° C. for 10 minutes. The activated transglutaminase as described above was added at amounts of 0.2, 0.4, 0.8 and 1.6 units and stirred for periods from 0 to 80 minutes. In the case of the control, the transglutaminase was not added or inactivated transglutaminase was added at an amount of 0.8 units. Viscosity of the solutions prepared thus was measured according to the content and reaction time of the transglutaminase. The viscosity was measured under conditions of a temperature of 50° C., a coning angle of 0.8° and a cone type of CP-40 using a Brookfield viscometer (LVTC-CP, Brookfield, USA).

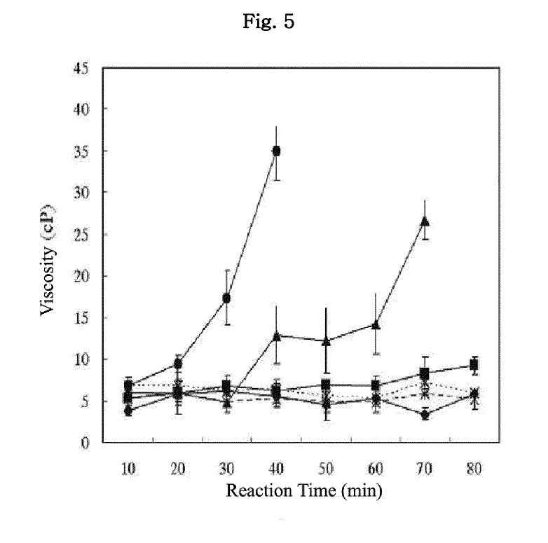

As a result, if the transglutaminase was not added or the inactivated transglutaminase was added, the viscosity of the film-forming composition was not increased with the passage of the reaction time. Meanwhile, if the transglutaminase was added, the film-forming composition comprising fish gelatin was increased as the reaction time and content of the transglutaminase increase (see FIG. 5).

### b) Examination of Cross-Linking Rate

The cross-linking rate of the film-forming composition was measured using a Bubnis & Ofner method (William. A, Bubnis and Clyde. M Ofner III, Analytical Biochemistry, 129-133, 1992). Absorbance of the film-forming composition was measured at a wavelength of 346 nm using a UV/VIS spectrophotometer Lambda 2S (Perkin Elmer, USA), and then the cross-linking rate was measured according to the following equation.

Cross-linking Rate(%)=[1−(Absorbance of Cross-linked Film/Absorbance of Uncross-linked Film)]×100

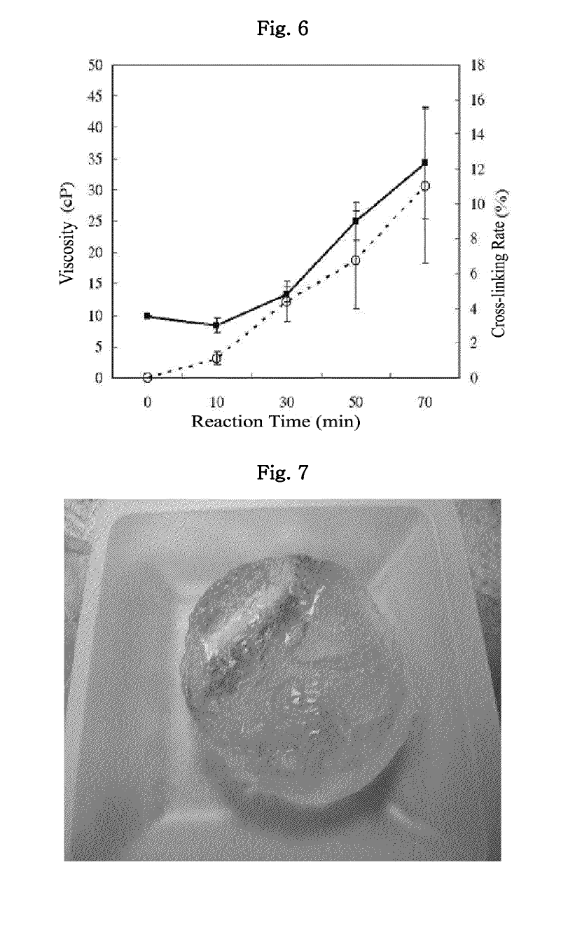

As a result, it was revealed that the cross-linking rate and viscosity of the film-forming composition were increased with passage of the reaction time after the addition of the transglutaminase, as shown in FIG. 6 (see FIG. 6).

### 3-2. Determination of Amount and Reaction Time of Transglutaminase for Hard Capsules

From the Example 3-1, it was revealed that the viscosity and cross-linking rate of the hard capsule composition comprising fish gelatin were increased by means of the addition of the transglutaminase. Then, in order to determine optimum amount and reaction time of the transglutaminase suitable for preparation of hard capsules, the hard capsules and films including various contents of transglutaminase on the basis of the total weight of the fish gelatin were prepared in the same manner as described in the Example 1 by the inventors, as listed in the following Table 3. Glycerol was used as the plasticizer, and the content of the plasticizer was used at a fixed content of 10% by weight on the basis of the total weight of the fish gelatin, which is within the optimum range of the plasticizer determined in the Example 2. The reaction time was 10-510 minutes and the irradiation was carried out per 10 minutes.

TABLE 3
Determination of Amount Reaction Time of Transglutaminase
Suitable for Preparation of Hard Capsules

| Fish Gelatin (wt % (w/v)) | 20 | | | | 25 | | | | 30 | | | | 35 | | | |
|---|---|---|---|---|---|---|---|---|---|---|---|---|---|---|---|---|
| Glycerol (wt %) | 10 | | | | | | | | | | | | | | | |
| Distilled Water (ml) | 100 | | | | | | | | | | | | | | | |
| Transglutaminase (units) | 0.1 | 0.6 | 1.0 | 1.4 | 0.125 | 0.75 | 1.3 | 1.8 | 0.15 | 0.9 | 1.5 | 2.1 | 0.175 | 1.1 | 1.8 | 2.5 |

| Reaction Time (min) | 10-510 | | | | | | | | | | | | | | | |
|---|---|---|---|---|---|---|---|---|---|---|---|---|---|---|---|---|
| Fish Gelatin (wt % (w/v)) | 40 | | | | 45 | | | | 50 | | | | 70 | | | |
| Glycerol (wt %) | 10 | | | | | | | | | | | | | | | |
| Distilled Water (ml) | 100 | | | | | | | | | | | | | | | |
| Transglutaminase (units) | 0.3 | 1.2 | 2.0 | 2.8 | 0.225 | 1.4 | 2.3 | 3.2 | 0.25 | 1.5 | 2.5 | 3.6 | 0.32 | 2.1 | 3.5 | 4.9 |

| | |
|---|---|
| Reaction Time (min) | 10-510 |

Contents of the added glycerol are based on the total weight of the fish gelatin.

As a result, it was revealed that the capsule was formed if the fish gelatin and the transglutaminase were added at the total weight of 30-50 g and a content of 0.1-5.0 units, respectively, based on 100 ml of the distilled water. If the content of the transglutaminase is less than 0.1 units, the capsules were not properly formed at 15-25° C. (it is impossible to present a final product (not shown) since the product is not made). Meanwhile, if the content of the transglutaminase exceeds 5 units, it was impossible to prepare hard capsules since the aqueous fish gelatin solution was all gelled within 30 minutes (see FIG. 7).

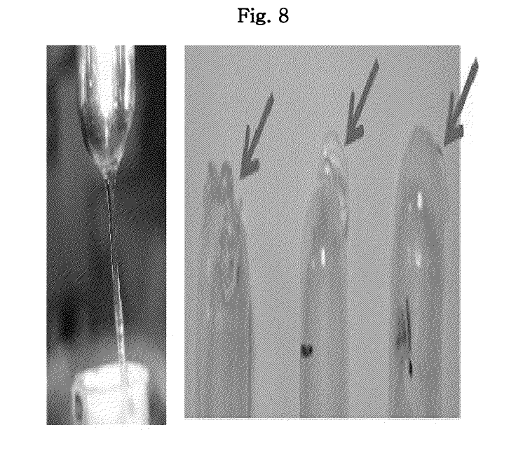

Also, it was shown that the reaction time of the transglutaminase was shortened with the increasing content of the transglutaminase. Preferably, the most suitable reaction time was shown to be 30 to 160 minutes. If the reaction time is less than 30 minutes, the capsule was formed at a thinner thickness than the thickness which is required for forming the capsule since the enzyme reaction was generally made at a low level, whereas if the reaction time is longer than 160 minutes, a tailing phenomenon occurred in the procedure of immersing the molding pin in the aqueous fish gelatin solution and pulling out the molding pin from the aqueous fish gelatin solution (see FIG. 8).

Accordingly, it was revealed that the content of the transglutaminase, which is suitable for preparation of the film and the capsules, was 0.1-5.0 units, and the optimum reaction time was 30-160 minutes.

### Example 4

### Preparation of Film and Hard Capsules Comprising Fish Gelatin

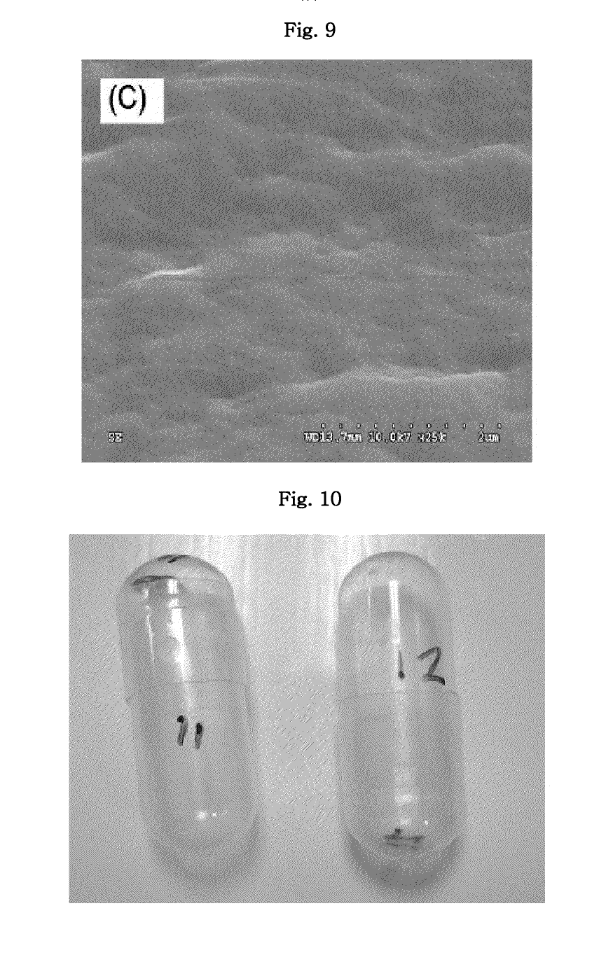

A film and hard capsules were prepared with fish gelatin in the same manner as described in the Example 1, as listed in the following Table 4. An SEM microphotograph enlarged and taken from a section of the resultant film was shown in FIG. 9. Also, the prepared hard capsules were shown in FIG. 10.

TABLE 4
Components and Content of Film-forming Composition for Hard
Capsules Comprising Fish Gelatin

| | |
|---|---|
| Fish Gelatin | 40% by weight (w/w) |
| Glycerol | 10% by weight |
| Distilled Water | 100 ml |
| Transglutaminase | 0.8 units |

Contents of the added glycerol are based on the total weight of the fish gelatin.

### Example 5

### Evaluation of Physical Properties of Hard Capsules Comprising Fish Gelatin

The hard capsules comprising fish gelatin prepared in the Example 4 were measured for physical properties such as tensile strength, elongation, oxygen permeability, moisture permeability, thermal property, colors and opacity. A fish gelatin capsule (hereinafter, referred to as FG only), a gelatin capsule comprising animal fish and an HPMC capsule were used as the controls, the fish gelatin capsule containing the transglutaminase prepared according to the method known in the Korean Patent No. 10-0483072.

### 5-1. Measurement of Tensile Strength and Elongation

Tensile strength and elongation of the hard capsules comprising fish gelatin were measured according to an ASTM standard D 882-88 (ASTM, 1989) method by using a physical property testing system (SATEC System, Inc, USA). The results are listed in the following Table 5.

TABLE 5
Measurement of Tensile Strength and Elongation

Mechanical Properties

| | Tensile Strength | Elongation |
|---|---|---|
| | (MPa) | (%) |
| FC only | 18.66 ± 0.54 | 137.71 ± 6.95 |
| Inventive Capsule | 60.97 ± 1.9 | 1.47 ± 0.05 |
| Comprising Fish Gelatin | | |
| Hard Capsule Comprising | 19.5 ± 1.6 | 122.0 ± 14.6 |
| Animal Gelatin | | |
| HPMC Hard Capsule | 19.9 ± 1.2 | 13.8 ± 4.2 |

From the above results, it was revealed that the hard capsules comprising fish gelatin prepared in the present invention have an excellent tensile strength, compared to the conventional hard capsules.

### 5-2. Oxygen Permeability

The oxygen permeability was measured according to an ASTM standard D 3985-81 (ASTM, 1989b) method by using an OX-Tran 2/60 O2 transmission tester (Mordern Control, Inc, USA) The results are listed in the following Table 6.

TABLE 6
Measurement of Oxygen Permeability

| | Oxygen Permeability |
|---|---|
| | (cc/m² · day) |
| FC only | 6.94 ± 1.33 |
| Inventive Capsule | 17.69 ± 3.08 |
| Comprising Fish | |
| Gelatin | |
| Hard Capsule | 1.15 ± 0.08 |
| Comprising Animal | |
| Gelatin | |
| HPMC Hard Capsule | 260 ± 11.3 |

From the above results, it was revealed that the hard capsules comprising fish gelatin prepared in the present invention have a high oxygen permeability, compared to the conventional animal hard capsules or fish gelatin capsules, but have a significantly low oxygen permeability, compared to the HPMC hard capsules. Therefore, it was confirmed that the hard capsules comprising fish gelatin of the present invention have a very excellent oxygen barrier property, compared to the HPMC hard capsule.

### 5-3. Moisture Permeability

The moisture permeability was measured using an MOCON Permatran-W3/31 Water Vapor Permeation Measurement System (Modern Controls Inc., USA). The results are listed in the following Table 7.

TABLE 7
Measurement of Moisture Permeability

| | Moisture |
|---|---|
| | Permeability |
| | (g · m/m² · day · atm) |
| FC only | 1.63 ± 0.30 |
| Inventive Capsule | 1.53 ± 0.22 |
| Comprising Fish Gelatin | |
| Hard Capsule Comprising | 21.88 ± 1.75 |
| Animal Gelatin | |
| HPMC Hard Capsule | 21.00 ± 4.38 |

From the above results, it was revealed that the hard capsules comprising fish gelatin prepared in the present invention have an ability to reduce damage of inside medicinal compounds by moisture since they have a low moisture permeability, compared to the conventional hard capsules comprising animal gelatin and the HPMC hard capsules.

### 5-4. Thermal Property

The thermal property of the hard capsules comprising fish gelatin according to the present invention was measured using a differential scanning calorimeter (TA Instruments, USA). A rising temperature was 10° C./min and a temperature period was within the range of 0 to 200° C. The results are listed in the following Table 8.

TABLE 8
Measurement of Thermal Property

| | Temperature (° C.) |
|---|---|
| FC only | 94.78 ± 7.34 |
| Inventive Capsule | 134.75 ± 4.11 |
| Comprising Fish Gelatin | |
| Hard Capsule Comprising | 87.03 ± 3.21 |
| Animal Gelatin | |
| HPMC Hard Capsule | 72.56 ± 2.26 |

From the above results, it was revealed that the hard capsules comprising fish gelatin prepared in the present invention have a high heat resistance, compared to the conventional hard capsules.

### 5-5. Colors

The colors of the hard capsules comprising fish gelatin according to the present invention were measured using a chromameter CR-400 (Minolta, Japan). Phosphor C was used as a power source and represented by CIE L*a*b* values. The results are listed in the following Table 9.

TABLE 9
Measurement of Colors

| | CIE L*a*b* | | |
|---|---|---|---|
| | L* | a* | b* |
| FC only | 95.56 ± 0.09 | −0.17 ± 0.01 | 3.17 ± 0.01 |
| Inventive Capsule | 95.48 ± 0.07 | −0.23 ± 0.03 | 3.47 ± 0.03 |
| Comprising Fish | | | |
| Gelatin | | | |
| Hard Capsule | 90.18 ± 0.27 | −2.65 ± 0.03 | 13.06 ± 0.25 |
| Comprising Animal | | | |
| Gelatin | | | |
| HPMC Hard Capsule | 90.82 ± 0.02 | −1.44 ± 0.11 | 4.06 ± 1.05 |

From the above results, it was revealed that the hard capsules comprising fish gelatin prepared in the present invention may have a better coloring effect since they have a good transparency, compared to the conventional hard capsules.

### 5-6. Opacity

The opacity of the hard capsules comprising fish gelatin according to the present invention was measured using a THWING-ALBERT digital opacimeter model 628 (THWING-ALBERT, USA) and represented by TAPPI opacity values. The results are listed in the following Table 10.

TABLE 10
Measurement of Opacity

| | Opacity |
|---|---|
| FC only | 1.43 ± 0.32 |
| Inventive Hard Capsules | 2.90 ± 0.00 |
| Comprising Fish Gelatin | |

From the above results, it was revealed that opacity of the hard capsules comprising fish gelatin prepared according to the method of the present invention is increased by the treatment of the transglutaminase.

### INDUSTRIAL APPLICABILITY

According to the present invention, the problems caused due to the characteristics of fish gelatin having a low gelling temperature were solved by employing transglutaminase in the preparation of hard capsules using fish gelatin. The method for preparing hard capsules using the film-forming composition for hard capsules comprising fish gelatin according to the present invention is more effective in aspects of the time and cost than the conventional methods since the film-forming composition of present invention may be dried at 15-25° C. Accordingly, the composition according to the present invention, its preparation method and the hard capsules and film comprising the composition may be useful to apply to a variety of industrial fields such as medicines, foods, etc.

1. A film-forming composition for hard capsules comprising fish gelatin, the composition being prepared by mixing 100 ml of distilled water with 20-70% by weight (w/v) of fish gelatin on the basis of 100 ml of the distilled water, adding 5-25% by weight of a plasticizer on the basis of the total weight of the fish gelatin and adding 0.1-5.0 units of transglutaminase.

2. The composition according to claim 1, wherein the plasticizer is selected from the group consisting of glycerol, sorbitol, polyethylene glycerol (PEG), mannitol and mantitol, and combinations thereof.

3. A method for preparing a film-forming composition for hard capsules, the method comprising:
(a) mixing fish gelatin with a distilled water at 50-90° C. and adding a plasticizer to the resultant mixture to dissolve the fish gelatin for 8-24 hours;
(b) cooling the solution prepared in the step (a) to 45-55° C. and adjusting pH of the solution to pH 6-8;
(c) adding transglutaminase to the solution prepared in the step (b) and stirring the solution at 45-55° C. for 30-160 minutes; and
(d) heating the solution prepared in the step (c) to 90-100° C. for 5-20 minutes and cooling the solution to 30-40° C.

4. The method according to claim 3, wherein the fish gelatin in the step (a) is mixed at an amount of 20-70% by weight (w/v), based on 100 ml of the distilled water.

5. The method according to claim 3, wherein the plasticizer in the step (a) is added at an amount of 5-25% by weight, based on the total weight of the fish gelatin.

6. The method according to claim 3, wherein the transglutaminase in the step (c) is added at an amount of 0.1-5.0 units.

7. A method for preparing hard capsules comprising fish gelatin, the method comprising:
immersing a molding pin in the film-forming composition for hard capsules prepared according to the method of claim 3; and
drying the molding pin at 15-25° C.

8. A film for hard capsules comprising the composition of claim 1.

9. Hard capsules comprising the composition of claim 1.
